(19) United States
(12) Patent Application Publication
(10) Pub. No.: US 2004/0224937 A1
(43) Pub. Date: Nov. 11, 2004

(54) Use of protein kinase C inhibitor for suppressing sustained slow postsynaptic excitation (SSPE)

(75) Inventors: John B. Furness, Fitzroy (AU); Martin J. Stebbing, Thornbury (AU)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/811,465

(22) Filed: Mar. 26, 2004

Related U.S. Application Data
(60) Provisional application No. 60/458,673, filed on Mar. 28, 2003.

(51) Int. Cl. A61K 31/553

(52) U.S. Cl. 514/211.1

(57) ABSTRACT

A novel use of an inhibitor of protein kinase C (PKC) for suppressing the sustained slow postsynaptic excitation (SSPE) caused by prolonged stimulation of synaptic inputs to intrinsic primary afferent neurons (IPANs), is provided. Since the generation of SSPE requires a phosphorylation step mediated by protein kinase C, PKC inhibitors, such as staurosporine, RO31-8220 or calphostin C is useful for a method for the treatment of a patient having a need to suppress the sustained slow postsynaptic excitation (SSPE) caused by prolonged stimulation of synaptic inputs to intrinsic primary afferent neurons (IPANs), such as intestinal hypersensitivity, irritable bowel syndrome (IBS), non-ulcer dyspepsia, or other conditions that may derive from long-term changes in the behavior of enteric neurons.

### RELATED APPLICATIONS

This application claims benefit of priority under 35 U.S.C. § 119(e) to U.S. Provisional Application No. 60/458,673, filed Mar. 28, 2003, which is incorporated herein in its entirety by reference.

### FIELD OF THE INVENTION

The present invention relates to a use of a protein kinase C inhibitor for suppressing the sustained slow postsynaptic excitation (SSPE) caused by prolonged stimulation of synaptic inputs to intrinsic primary afferent neurons (IPANs) of the gastrointestinal tract.

### BACKGROUND OF THE INVENTION

Long-term changes in neuronal excitability follow presynaptic excitation in the mammalian central nervous system and in invertebrates (Bliss, T V P, et al., Nature 361: 31-39, (1993); Kandel, E R, Science 294: 1030-1038 (2001)). In the central nervous system, the most commonly studied phenomenon is long-term potentiation (LTP) that occurs most notably in the hippocampus. The major invertebrate model is Aplysia, in which long-term facilitation follows nerve stimulation or agonist action (Kandel, E R, Science 294: 1030-1038 (2001)).

A long-term increase in excitability induced by presynaptic excitation has recently been described in the mammalian enteric nervous system (Clerc, N, et al., Neuroscience 90: 279-289, (1999); Alex G, et al., Neuroscience 104: 263-269 (2001); Alex G, et al., Neuroscience 110: 361-373 (2002)). This event, called sustained slow postsynaptic excitation (SSPE) is induced by prolonged (usually 4 minutes or more) stimulation of synaptic inputs to enteric neurons. The stimulation causes periods of increased neuron excitability, associated with depolarization and increased input resistance that can last up to 4 hours beyond the period of stimulation of synaptic inputs. The SSPE occurs in the intrinsic primary afferent neurons (IPANs) of the myenteric ganglia, but not in interneurons or motor neurons (Alex, G. et al., Neuroscience 110: 361-373 (2002)).

The cellular mechanisms for LTP in the mammalian central nervous system and long-term excitability changes in molluscan neurons have been extensively investigated, and have been shown to depend on protein phosphorylation (Malinow, R, et al., Nature 335: 820-824 (1988); Wang, J H, et al., Proc. Natl. Acad. Sci. USA 89: 2576-2580 (1992); Bliss, T V P, et al., Nature 361: 31-39 (1993); Blitzer, R D, et al., Science 280: 1940-1943 (1998); Manseau, F, et al, J. Neurophysiol. 79: 1210-1218 (1998)), but nothing is known about the mechanisms underlying SSPE.

As the initiating neurons for enteric reflexes, the IPANs are strategically placed to influence the intensity of enteric reflexes if their excitability is changed, for example during the SSPE. Moreover, structural evidence suggests that there could be interaction between IPANs and the processes of extrinsic primary afferent neurons (Mazzia C, et al., Neuroscience 80: 925-937 (1997)). Thus, changes in excitability of IPANs could influence sensory signaling from the gut by changing the intensity of intrinsic reflexes or through connections with extrinsic primary afferent neurons. For these reasons, it has been suggested that changes in excitability of IPANs may be involved in the development of intestinal hypersensitivity and changed motility in irritable bowel syndrome (Mayer, E A, et al., Gastroenterology 116: 1250-1252 (1999); Bueno L, et al., Am. J. Physiol. 278: G670-G676 (2000)). However, there have been no reports in regard to a dependence of the SSPE on protein kinase activity.

If activation of protein kinase is involved in the initiation of the SSPE, the protein kinase may be a target for drugs that could be used to treat intestinal hypersensitivity and/or irritable bowel syndrome (IBS) as well as other conditions that may derive from long-term changes in the behavior of enteric neurons.

Thus, bearing this in mind, it is necessary to investigate the roles of various protein kinases in SSPE and to provide a new method for treating intestinal hypersensitivity and IBS by suppressing the SSPE that occurs in IPANs.

### SUMMARY OF THE INVENTION

In a broad aspect, the present invention relates to novel use of an inhibitor of protein kinase C (PKC) for suppressing the sustained slow postsynaptic excitation (SSPE) caused by prolonged stimulation of synaptic inputs to intrinsic primary afferent neurons (IPANs).

In another aspect, the present invention relates to novel use of a protein kinase C (PKC) inhibitor in the manufacture of a medicament for the treatment of a disease selected from the group consisting of intestinal hypersensitivity, irritable bowel syndrome (IBS), and other conditions that may derive from long-term changes in the behavior of enteric neurons.

### BRIEF DESCRIPTION OF THE FIGURES

FIGS. 1A-D show the effect of staurosporine (1 μM) on the sustained slow postsynaptic excitation (SSPE) in response to stimulation of synaptic inputs to myenteric intrinsic primary afferent neurons (IPANs). Incoming fibers were stimulated at 1 Hz for 4 min at the bars. Continuous records of excitability (FIG. 1A), measured as the number of action potentials evoked by a 500 ms depolarizing pulse of 100 pA applied through the recording electrode, membrane potential (FIG. 1B) and input resistance (FIG. 1C) of an IPAN are shown. Nerve stimulation caused an increase in excitability, depolarized the neuron and increased its input resistance. After the application of staurosporine (1 μM), the stimulation had no effect on excitability. Membrane potential and input resistance recovered towards their pre-stimulus values. (FIG. 1D) shows records of the responses caused by intracellular 500 ms depolarizing pulses before stimulation (a), at the peak of SSPE (b), and after the action of staurosporine (c). These records were taken at the points indicated in (A).

FIG. 2 shows effects of kinase inhibitors on SSPE. In each group of bars, the first two (filled bars) represent the excitability increases of IPANs in response to two stimuli (each 1 Hz for 4 min, applied to presynaptic inputs), and the third (unfilled) bar is the excitability change in the presence of the kinase inhibitor. Leftmost bars show effect of staurosporine (1 μM). Staurosporine decreased the peak change in excitability by about 75%, which is significantly different to control (n=6, P<0.01). Middle left bars show effect of H89 (1 μM). H89 had no effect on the SSPE (n=7, >0.9). Middle right bars show effect of RO31-8220 (3.3 μM). RO31-8220 reduced the degree to which the excitability increased during the SSPE (n=6, P<0.1). Rightmost bars show effect of calphostin C (1 μM). After the application of calphostin C, the increase in excitability caused by stimulation of synaptic inputs was blocked; the excitation after calphostin C was significantly different from control (n=8, P<0.01). In fact, the excitability was less after than before the stimulus in the presence of calphostin C.

FIGS. 3A-B show lack of effect of H89 (1 μM) on SSPE in response to stimulation of synaptic inputs to myenteric IPANs. Presynaptic fibers were stimulated at 1 Hz for 4 min at the bars. (FIG. 3A) shows a continuous record of excitability, measured as the number of action potentials evoked by a 500 ms depolarizing pulse of 100 pA applied through the recording electrode. Nerve stimulation caused an increase in excitability in two tests applied in the absence of H89, and also after the preparation was exposed to H89. (FIG. 3B) shows records of the responses caused by intracellular 500 ms depolarizing pulses before stimulation(a), at the peak of the first SSPE (b) and during SSPE after exposure of the tissue to H89(c). These records were taken from the data used to construct record (FIG. 3A), at the points indicated on that record. H89 had no effect on SSPE.

FIGS. 4A-B show the effect of calphostin C (1 μM) on the SSPE in response to stimulation of synaptic inputs to myenteric IPANs. Presynaptic fibers were stimulated at 1 Hz for 4 min at the bars. (FIG. 4A) shows a record of excitability, measured as the number of action potentials evoked by a 500 ms depolarizing pulse of 100 pA, applied through the recording electrode. The second SSPE was larger than the first, although the excitability increase caused by the first stimulus had not completely subsided. In the presence of calphostin C, stimulation did not cause an increase in excitability. In fact, excitability was less after than before the stimulus. (FIG. 4B) shows records of the responses caused by intracellular 500 ms depolarizing pulses before stimulation (a), during the period after the second stimulus (b) and after the action of calphostin C (c). These records were taken from the data used to construct record (FIG. 4A), at the points indicated on that record.

FIGS. 5A-D show an example of the effect of phorbol dibutyrate (PDBu, 1 μM) on the excitability of an IPAN in a myenteric ganglion. PDBu increased the excitability of the neuron (FIG. 5A), caused a depolarization of about 8 mV (FIG. 5B), and increased input resistance by about 100 MΩ (FIG. 5C). (FIG. 5D) shows responses of the neuron to intracellular current injection (100 pA) at (a), (b), and (c) in trace (FIG. 5A).

FIGS. 6A-C show immunoreactivity for PKC isoforms and co-localization with immunoreactivity for calbindin, a marker of intrinsic primary afferent neurons, in whole amounts of the myenteric plexus. Of the PKC isoforms tested, three, PKCγ(FIG. 6A), PKCε(FIG. 6B) and PKCλ(FIG. 6C) were co-localized with calbindin in myenteric neurons of the guinea pig small intestine. PKCγ did not occur in other neurons, whereas PKCε and PKCλ were both in a majority of neurons. Neurons with both a PKC isoform and calbindin immunoreactivity are indicated by the filled arrows, and neurons that were reactive only for the PKC isoform are indicated by arrows with open ends. Scale bars: 50 μm.

### DETAILED DESCRIPTION OF THE INVENTION

The inventors have investigated the effects of kinase inhibitors on the sustained slow postsynaptic excitation (SSPE) caused by prolonged stimulation of synaptic inputs to intrinsic primary afferent neurons in the small intestine of the guinea pig, the effects of the PKC stimulant, phorbol dibutyrate, on these neurons, and the immunohistochemical localization of PKC isoforms in enteric neurons. The SSPE was substantially reduced by the broad spectrum kinase inhibitor staurosporine (1 μM), by the PKC inhibitors, RO31-8220 (3.3 μM) and calphostin C (1 μM), but not by the PKA inhibitor H89 (1 μM). Phorbol dibutyrate (1 μM) caused excitability increases, membrane depolarization and increased input resistance that mimicked the SSPE. PKCγ, PKCε and PKCλ, but not PKCα, PKCβ, PKCδ, PKCη, PKCθ or PKCι, were located in intrinsic primary afferent neurons that were identified by their shapes and calbindin immunoreactivity in double stained preparations.

The inventors have now found that the generation of SSPE requires a phosphorylation step mediated by protein kinase C.

According to one aspect of the present invention, there is provided a use of an inhibitor of protein kinase C (PKC) for suppressing the sustained slow postsynaptic excitation (SSPE) caused by prolonged stimulation of synaptic inputs to intrinsic primary afferent neurons (IPANs). The PKC may be selected from the group consisting of PKCγ, PKCε, and PKCλ. In particular, the PKC may be PKCγ. The inhibitor may be selected from the group consisting of staurosporine, RO31-8220, calphostin C, and compounds with similar pharmacological profiles.

According to another aspect of the invention, there is provided a use of a protein kinase C (PKC) inhibitor in the manufacture of a medicament for the treatment of a disease selected from the group consisting of intestinal hypersensitivity, irritable bowel syndrome (IBS), non-ulcer dyspepsia, and other conditions that may derive from long-term changes in the behavior of enteric neurons. The PKC inhibitor may be selected from the group consisting of staurosporine, RO31-8220, calphostin C, and compounds with similar pharmacological profiles. RO31-8220 is the protein kinase C inhibitor, 3-[1-[3-(Amidinothio)propyl-1H-indol-3-yl]-3-(1-methyl-1H-indol-3-yl)maleimide Bisindolylmaleimide IX, methanesulfonate (commericially available from CALBIOCHEM, San Diego, Calif.).

According to a further aspect of the invention, there is provided a method for the treatment of a patient having a need to suppress the sustained slow postsynaptic excitation (SSPE) caused by prolonged stimulation of synaptic inputs to intrinsic primary afferent neurons (IPANs), comprising administering to the patient a therapeutically effective amount of a protein kinase C (PKC) inhibitor. The PKC inhibitor may be selected from the group consisting of staurosporine, RO31-8220, calphostin C, and compounds with similar pharmacological profiles.

The PKC may be PKCγ, and the inhibitor may be a specific inhibitor of PKCγ.

SSPE is initiated in intrinsic primary afferent neurons (IPANs) of the guinea pig small intestine by a single train of low frequency stimulation of presynaptic inputs (Clerc, N, et al., Neuroscience 90: 279-289 (1999); Alex, G, et al., Neuroscience 110: 361-373 (2002)). It can be considered to have initiation, facilitation and maintenance phases. During and after the first stimulation, the excitability of the IPANs slowly increases, they depolarize, and their input resistance increases markedly, often by over two fold. This magnitude of increase, accompanied by depolarisation, means that K⁺ channels are closed. The second or third of successive stimulus trains often elicits a greater increase in excitability than does the first train (facilitation). The maintenance phase, caused by longer trains of stimulation, or the accumulated effects of several trains, involves enhanced excitability of these neurons that can outlast stimulation by several hours (Clerc, N, et al., Neuroscience 90: 279-289 (1999)).

In the present study, we have found that SSPE is substantially reduced by the broad spectrum kinase inhibitor, staurosporine (1 μM). However, it was not affected by the PKA antagonist, H89 (1 μM). The PKC inhibitor, RO31-8220 (3.3 μM) also suppressed SSPE. However, there was evidence that at 10 μM this compound reduces the excitability of enteric axons. Calphostin C (1 μM) also reduced the amplitude of SSPE, and once SSPE had been initiated, further facilitation was prevented by calphostin C. These data all suggest that activation of PKC is involved in the initiation of SSPE. However, the excitability increase that had been induced prior to the addition of calphostin C was not reversed, and generally declined only slowly, suggesting that the maintenance phase of SSPE is not dependent on continuing activation of PKC.

The kinase inhibitors had little effect on the resting properties of the intrinsic sensory neurons. Moreover, they did not change action potential characteristics or cause a deterioration of the neurons in the time span of these experiments. This suggests that in their resting state, in isolated preparations removed from the animal, activity of PKC does not contribute to the excitability of IPANs. However, the PKC stimulant, phorbol ester (PDBu), caused the neurons to depolarize, increase input resistance and increase excitability, changes that are very similar to the SSPE evoked by stimulation of synaptic inputs at 1 Hz for 4 min or more.

As the initiating neurons for enteric reflexes, the IPANs are strategically placed to influence the intensity of enteric reflexes if their excitability is changed, for example during the SSPE. Moreover, structural evidence suggests that there could be interaction between IPANs and the processes of extrinsic primary afferent neurons (Mazzia, C, et al., Neuroscience 80: 925-937 (1997)). Thus, changes in excitability of IPANs could influence sensory signaling from the gut by changing the intensity of intrinsic reflexes or through connections with extrinsic primary afferent neurons. For these reasons, it has been suggested that changes in excitability of IPANs may be involved in the development of intestinal hypersensitivity and changed motility in irritable bowel syndrome (Mayer, E A, et al., Gastroenterology 116: 1250-1252 (1999); Bueno, L, et al., Am. J. Physiol. 278: G670-G676 (2000)). If this is the case, PKC might be a target for drugs that could be used to treat these or other conditions that may derive from long-term changes in the behavior of enteric neurons. In this respect, it is notable that PKCγ is located in IPANs, and perhaps not in other neuron types. Immunoreactivity for PKCγ was found in calbindin immunoreactive neurons, which are known to be about 80-85% of the IPANs in the myenteric ganglia (Iyer, V, et al., J. Auton. Nerv. Syst. 22: 141-150 (1988); Song, Z M, et al., Neurosci. Lett. 129: 294-298 (1991)). An observation that PKCγ is confined to IPANs, suggests that specific inhibitors of this isoform may have therapeutic potential in IBS.

The targets for PKC in causing SSPE are not known, but, directly or indirectly, SSPE must cause a change in the opening probability or conductance of ion channels. The ion channels could in fact be direct targets for PKC-dependent phosphorylation.

Other characteristics and advantages of the present invention will be seen in the Examples below related to research activities that led to the demonstration and the characterization of use or method of the present invention, and in which reference will be made to the annexed figures.

### EXAMPLES

### Methods and Materials

Guinea pigs from the inbred Hartley strain colony of the Department of Anatomy and Cell Biology at the University of Melbourne were used. All efforts were made to minimize animal suffering and numbers of animals used. The experiments conformed to National Health and Medical Research Council of Australia guidelines and were approved by the University of Melbourne Animal Experimentation Ethics Committee. Guinea pigs of either sex (180-250 g) were stunned by a blow to the head and killed by severing the carotid arteries and spinal cord, and segments of ileum were removed into physiological saline. The segments were placed in a recording dish lined with silicone elastomer, opened along the line of the mesenteric attachment and pinned flat under moderate tension with the mucosa uppermost. During dissection, the tissue was immersed in physiological saline (composition in mM: NaCl 118, KCl 4.8, NaHCO₃ 25, NaH₂PO₄ 1.0, MgSO₄ 1.2, glucose 11.1, CaCl₂ 2.5) and kept at room temperature. The mucosa, submucosa and circular smooth muscle were removed to expose the myenteric plexus. The recording dish (volume 4 mL) was then placed on the stage of an inverted microscope and continuously superfused (4 mL/min) with physiological saline that had been preheated to yield a bath temperature of 35-37° C. The superperfusion solution was bubbled with 95% O₂ and 5% CO₂ and contained nicardipine (3 μM) and hyoscine (1 μM) to inhibit muscle movement. The tissue was equilibrated with the perfusate for 1-2 h before recording was commenced.

Individual neurons were impaled using conventional intracellular microelectrodes (resistance, 100-220 mΩ) filled with 1 M KCl. Signals were amplified using an AxoClamp 2B amplifier (Axon instruments, Foster City, Calif., USA), digitized at 5-20 kHz and stored using PC-based data acquisition software (Axoscope 8.0). Measurements were made after allowing the impalements to stabilize for at least 15 min without applying intracellular holding current. Neurons were identified as AH cells based on the presence of a hump on the falling phase of the action potential and, in most cells, a late after-hyperpolarising potential following the action potential. Stimuli were applied to inter-ganglionic connectives at a frequency of 1 Hz, via a fine tungsten stimulating electrode (tip diameter<50 μm) that was placed on a circumferentially located internodal strand adjacent to the ganglion. Pulses were 0.1 ms in duration and <0.3 mA in intensity.

Neuronal excitability was assessed, by injecting 500 ms depolarizing current pulses (0.05-0.2 nA). The depolarizing pulses were delivered at intervals of 40 s. The mean excitability was evaluated by averaging the numbers of action potentials evoked by the 6 depolarizing current pulses for periods of 4 min, prior to the 1 Hz stimulus, around the peak of the response, and for a period of 4 min after the end of the stimulus. To determine input resistance (Rᵢₙ), small hyperpolarizing current pulses (duration 100 ms; intensity<0.02 nA) were injected before each depolarizing pulse. The membrane potential (MP) was sampled each 40 s. MP, Rᵢₙ, and numbers of action potentials in response to depolarizing pulses were determined.

Immunoreactivity for PKC isoforms was localized in whole amounts of myenteric plexus attached to the longitudinal muscle. Segments of ileum were placed in phosphate buffered saline (PBS; 0.15M NaCl in 0.01M sodium phosphate buffer, pH 7.2) to which was added nicardipine (10⁻⁶ M) in order to prevent tissue contraction. Tissue was then opened and cleaned of contents, pinned tautly on balsa board, mucosa side down, and immersed in 2% formaldehyde plus 0.2% picric acid in 0.1 M sodium phosphate buffer, pH 7.0, at 4° C. overnight. The next day, tissue was cleared of fixative with 3×10 min washes in dimethylsulfoxide, followed by 3×10 min washes in PBS. Tissue was stored in PBS containing sodium azide (0.1%) at 4° C.

Following fixation and clearing, the tissue was dissected to prepare wholemounts of longitudinal muscle plus the myenteric plexus. Preparations were incubated in a 10% solution of normal horse serum plus 1% TritonX-100 in PBS for 30 min at room temperature, prior to exposure to antisera against PKC isoforms as listed in the following Table 1:

TABLE 1
Primary antisera used: BD = BD Biosciences,
San Jose, Ca, USA

| | | | Code and |
|---|---|---|---|
| | | | source or |
| Antiserum | Dilution | Host | reference |
| PKCγ | 1:250 | Mouse | BD |
| PKCλ | 1:100 | Mouse | BD |
| PKCε | 1:500 | Mouse | BD |
| PKCα | 1:500 | Mouse | BD |
| PKCβ | 1:100, 1:250 | Mouse | BD |
| PKCδ | 1:250, 1:500 | Mouse | BD |
| PKCη | 1:100, 1:250 | Mouse | BD |
| PKCθ | 1:100, 1:250 | Mouse | BD |
| PKCι | 1:100, 1:250 | Mouse | BD |
| Calbindin | 1:800 | Sheep | PES1: (Furness |
| | | | et al. 1989) |

Following incubation in primary antibodies for two or three nights at 4° C. in a humid chamber, preparations were given 3×10 min washes in PBS and then incubated for 1 hr at room temperature with appropriate secondary antibodies as listed in the following Table 2:

TABLE 2
Secondary antisera used. Suppliers: Amersham Pty Ltd,
Melbourne, Australia; Vector Laboratories, Burlingame,
Ca, USA

| Antibody and Label | Dilution | Source |
|---|---|---|
| Horse anti-Mouse | 1:100 | Vector |
| Biotin | | |
| Streptavidin | 1:400 | Amersham |
| TexasRed | | |
| Donkey anti-sheep | 1:50 | Amersham |
| FITC | | |

Double labeling was achieved using combinations of antisera (Table 1). Following incubation in primary antisera, tissue was given 3×10 min washes in PBS and then incubated in a mixture of secondary antibodies (see Table 2). A further 3×10 min washes in PBS were made before tissue was mounted in glycerol buffered with 0.5 M sodium carbonate buffer (pH 8.6).

Preparations were examined on a Zeiss Axioplan microscope equipped with the appropriate filter cubes for discriminating between fluorescein isothiocyanate (FITC) and Streptavidin-Texas Red (STR) fluorescence. The inventors used filter set 10 for FITC (450-490 nm excitation filter and 515-565 nm emission filter), and filter set 00 for STR (530-585 nm excitation filter and 615 nm emission filter). Images were recorded using a SpotRT cooled charge-coupled device camera and SpotRT 3.2 software (Diagnostic Instruments, Sterling Heights, Mich.). Preparations were also analyzed by confocal microscopy on a Biorad MRC 1024 confocal scanning laser system installed on a Zeiss Axioplan 2 microscope. The system had a krypton/argon laser for differential visualization of the fluorophores using 488 nm excitation filter and 522/535 mm emission filter for FITC, and 568 nm excitation filter and 605/632 nm emission filter for STR. The images were 512×512 pixels and the setting for nominal optical section thickness was 0.5 μm. Immunoreactive cells were scanned as a series of optical sections with a center to center spacing of 0.2 μm. Confocal images were collected using Biorad Lasersharp processing software. Images were further processed using Confocal Assistant, Corel PhotoPaint and Corel Draw software programs.

The compounds used were nicardipine (Sigma-Aldrich, Sydney, Australia), hyoscine (Sigma-Aldrich), staurosporine, H89, RO31-8220, calphostin C, phorbol 12, 13 dibutyrate (Sigma-Aldrich).

The results are given as means±SEM. The results were analyzed by ANOVA.

### Example 1

Effect of Staurosporine on Excitability, Membrane Potential, and Input Resistance

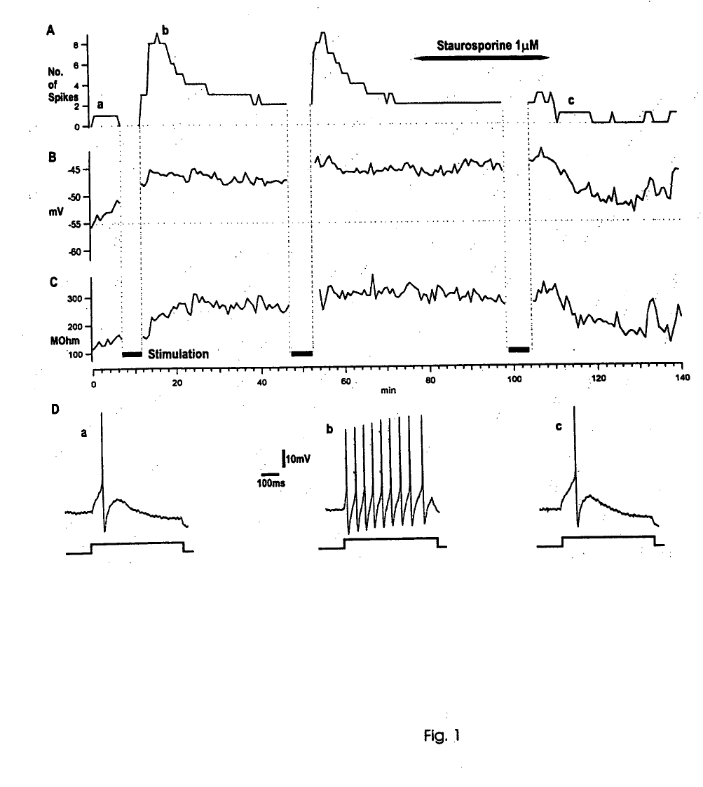

Neurons were identified as AH neurons by a slow after-hyperpolarizing potential (AHP) following the action potential. This type of neuron responded to 4 minutes stimulation of presynaptic inputs at 1 Hz with a prolonged increase in excitability, membrane depolarization and increased input resistance (FIG. 1). This prolonged excitation, that continues beyond the end of stimulation, has been previously described as sustained slow postsynaptic excitation (SSPE; Clerc, N, et al., Neuroscience 90: 279-289 (1999)). During the stimulus train, antidromic action potentials were often recorded in response to each stimulus pulse.

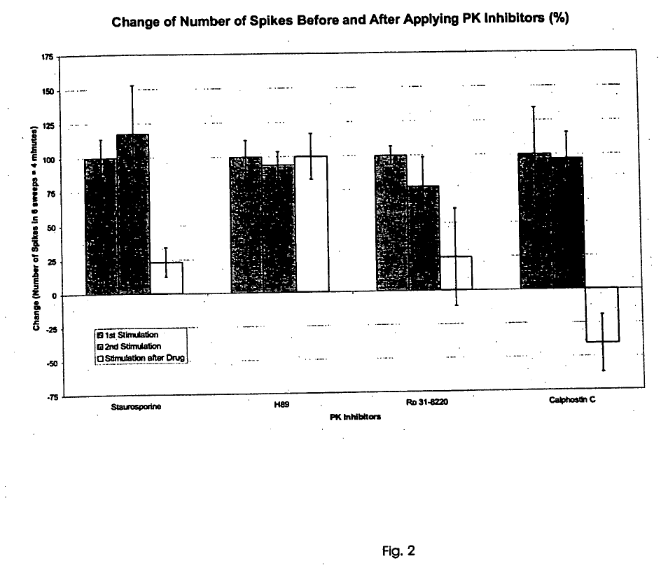

Staurosporine (1 μM) was added to the bath solution following two periods of stimulation that each generated a SSPE, and after the excitability increase associated with the second SSPE had subsided (FIG. 1). After 15-20 min of exposure to staurosporine, presynaptic inputs to the AH neurons were again stimulated. The addition of staurosporine did not cause any change in excitability, membrane potential or input resistance during this 15 min. However, stimulation of presynaptic inputs for 4 min at 1 Hz in the presence of staurosporine elicited a significantly smaller or no increase in neuronal excitability (FIGS. 1, 2). The excitability following stimulation was significantly different in the presence of staurosporine than before the compound was applied (n=6, P<0.01). In some cases, the membrane resistance and input resistance declined after the stimulation, which was 20-30 min after the application of staurosporine.

### Example 2

Effect of H89 on Excitability, Membrane Potential, and Input Resistance

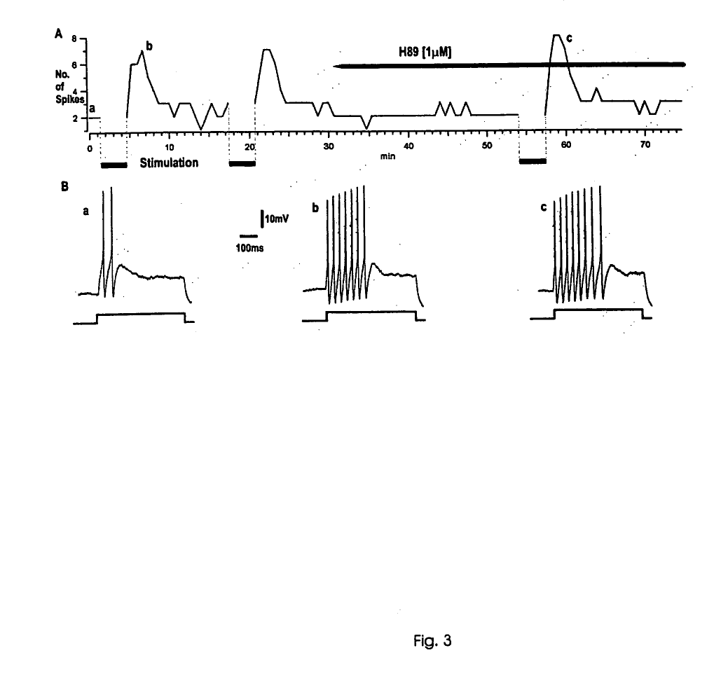

The PKA inhibitor, H89 (1 μM) was used in experiments that followed the same protocol used in the experiments with staurosporine. There were no changes in excitability, membrane potential or input resistance of AH neurons when preparations were exposed to H89 for 15 to 25 min. Responses to stimulation of presynaptic inputs for 4 min at 1 Hz were not changed by H89 (FIG. 3, n=7, P>0.9).

### Example 3

Effect of RO031-8220 on Excitability, Membrane Potential, and Input Resistance

The PKC inhibitor RO31-8220 was tested at 1, 3.3 and 10 μM. At 10 μM the antidromic action potentials were sometimes suppressed, but could be restored by increasing the stimulus strength. This suggests that RO31-8220 decreases the excitability of axons of enteric neurons at this concentration, which was therefore not used to evaluate effects of the drug on the SSPE. RO31-8220 (3.3 μM) did not cause a depression of antidromic action potential generation. However, it did cause a reduction in the increase of AH neuron excitability associated with SSPE (n=6, P<0.1), without affecting the excitability, membrane potential or input resistance during 15 min exposure before presynaptic inputs were stimulated. SSPE was not reduced by RO31-8220 (1 μM).

### Example 4

Effect of Calphostin on Excitability, Membrane Potential, and Input Resistance

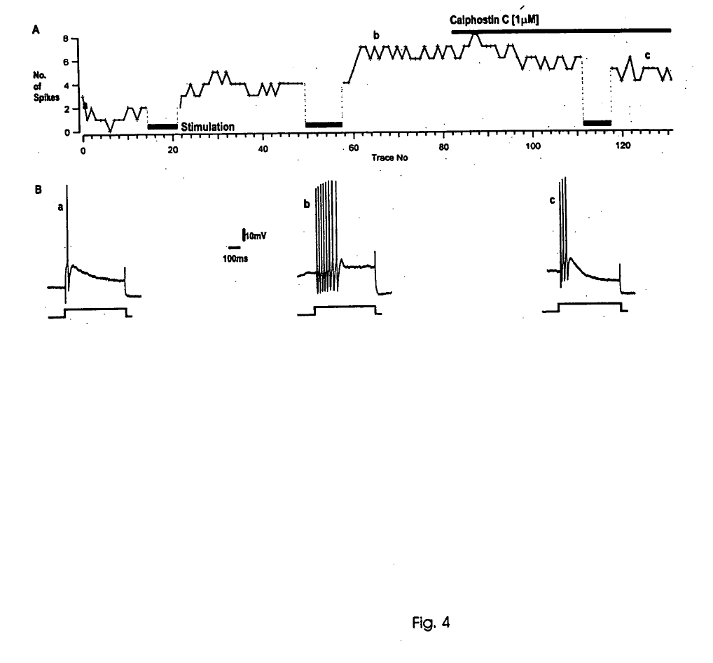

Calphostin C (1 μM) was applied to the preparations after two SSPEs had been evoked. In some of these experiments, the drug was added before the second SSPE had fully subsided. Calphostin C did not affect the excitability, membrane potential or input resistance prior to stimulation of presynaptic inputs. However, calphostin substantially reduced the excitability increase associated with the SSPE (FIGS. 2 and 4, n=8, P<0.01). In fact, if the excitability associated with the control SSPE, prior to addition of calphostin C to the organ bath, had not subsided, stimulation in the presence of calphostin C was associated with lower excitability after the stimulation than before it (FIG. 4). Calphostin C, added after the SSPE had been enhanced by successive stimuli, did not restore excitability to its pre-stimulus level (FIG. 4).

### Example 5

Effect of Phorbol Dibutyrate on Excitability, Membrane Potential, and Input Resistance

Phorbol dibutyrate (PDBu), 0.2-1 μM, was added to the bath solution for 5-10 min.

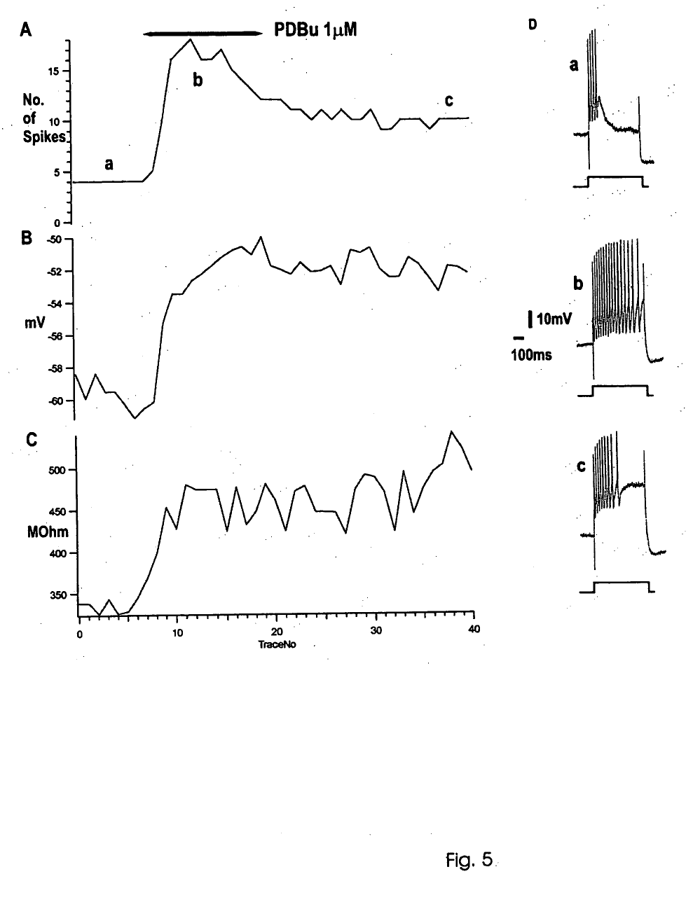

This caused changes very similar to SSPE (FIG. 5). At 1 μM, PDBu increased the number of action potentials elicited by a 500 ms depolarizing pulse by zz±xx action potentials, depolarized the membrane by zz±xx mV and increased input resistance by zz±xx (Quantitative data to come). The effect of PDBu continued for 10 min or more beyond the washing out of the compound. During exposure to PDBu, the amplitude of the AHP was decreased, as occurs during SSPE (Clerc et al., 1999).

### Example 6

Immunoreactivity for PKC Isoforms and Colocalization with Immunoreactivity for Calbindin, a Marker of Intrinsic Primary Afferent Neurons, in Whole Amounts of the Myenteric Plexus

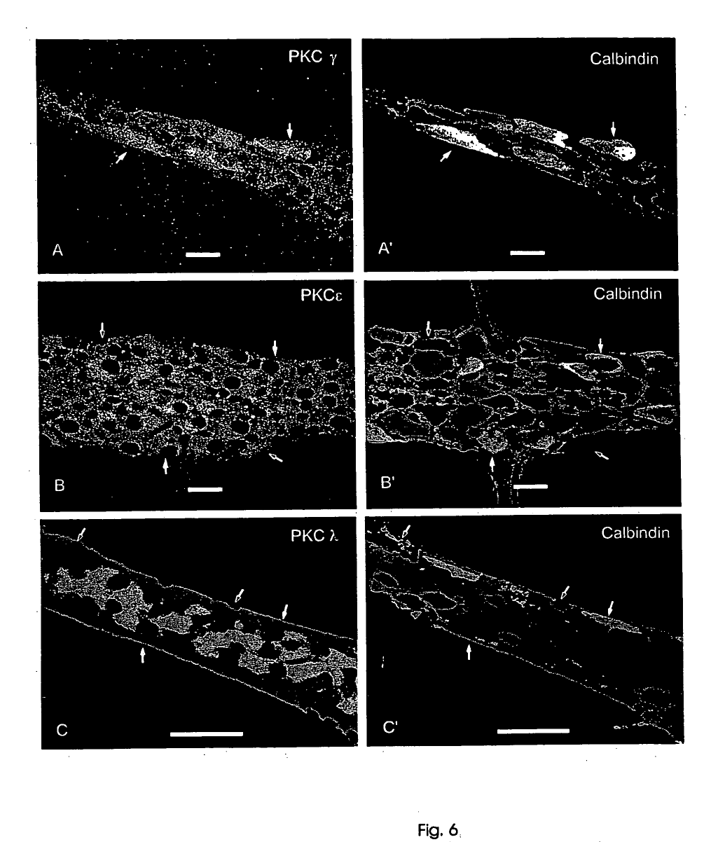

Immunoreactivity for PKC isoforms, PKCα, PKCβ, PKCγ, PKCδ, PKCε, PKCη, PKCθ, PKCι, and PKCλ was investigated in whole amounts of the myenteric plexus. The preparations were double stained with calbindin to reveal the AH neurons (Iyer, V, et al., J. Auton. Nerv. Syst. 22: 141-150 (1988)). Three isoforms, PKCγ, PKCε, and PKCλ were found in calbindin immunoreactive neurons (FIG. 6). These were large round or oval neurons, several of which were found in each ganglion. In the case of PKCγ, all immunoreactive neurons were also calbindin immunoreactive, and, conversely, all calbindin immunoreactive neurons had PKCγ immunoreactivity (FIG. 6A). PKCε immunoreactivity occurred in the majority of nerve cells, including both large nerve cells and small nerve cells (FIG. 6B). All nerve cells with calbindin immunoreactivity were also PKCε immunoreactive. PKCλ was also in the majority of neurons, including those that were calbindin immunoreactive (FIG. 6C). Antibodies against the PKC isoforms β, δ, η, η, θ bound to some myenteric neurons. No double labeling studies with calbindin were conducted, because the immunoreactivities with antibodies against these isoforms were very weak.

All references cited herein, including patents and patent applications, are hereby incorporated by reference.

It will be appreciated that the foregoing is provided by way of example only and modification of details may be made without departing from the scope of the invention.

The claimed invention is:

1. A method for the treatment of a patient having a need to suppress the sustained slow postsynaptic excitation caused by prolonged stimulation of synaptic inputs to intrinsic primary afferent neurons, comprising administering to the patient a therapeutically effective amount of a protein kinase C (PKC) inhibitor

2. The method of claim 1, wherein said PKC is selected from the group consisting of PKCγ, PKCε, and PKCλ.

3. The method of claim 1, wherein said PKC is PKCγ.

4. The method of claim 1, wherein said inhibitor is a selective inhibitor of PKCγ.

5. The method of claim 1, wherein said inhibitor is selected from the group consisting of staurosporine, RO31-8220, and calphostin C.

6. The method of any one of claims 1-5, wherein the patient is suffering from a disease selected from the group consisting of intestinal hypersensitivity, irritable bowel syndrome, non-ulcer dyspepsia, and other conditions that may derive from long-term changes in the behavior of enteric neurons.
